(19) United States
(12) Patent Application Publication
(10) Pub. No.: US 2018/0038841 A1
(43) Pub. Date: Feb. 8, 2018

(54) COMPONENT BASED ON A STRUCTURABLE SUBSTRATE WITH A MEMBRANE STRUCTURE HAVING THREE-DIMENSIONAL PORES IN THE NM RANGE AND SEMICONDUCTOR TECHNOLOGY METHOD FOR MANUFACTURING SAME

(71) Applicant: FRAUNHOFER-GESELLSCHAFT ZUR FÖRDERUNG DER ANGEWANDTEN FORSCHUNG E.V., München (DE)

(72) Inventors: Thomas LISEC, Itzehoe (DE); Eric NEBLING, Pinneberg (DE); Stefan KUBICK, Berlin (DE); Steffen RUPP, Stuttgart (DE)

(21) Appl. No.: 15/523,594

(22) PCT Filed: Oct. 21, 2015
(86) PCT No.: PCT/EP2015/074416

§ 371 Date: Oct. 27, 2017

(30) Foreign Application Priority Data
Oct. 31, 2014 (DE) 10 2014 115 884.9
Jan. 30, 2015 (DE) 10 2015 101 425.4

(51) Int. Cl. G01N 33/487; G01N 27/447

(52) U.S. Cl. CPC G01N 33/48721; G01N 27/44791

(57) ABSTRACT

The invention relates to a component, comprising a carrier made of a structurable material with at least one continues opening which is closed by a porous membrane, characterized in that the porous membrane protrudes from the surface of the component surrounding the continuous opening. In some embodiments, the component further comprises a carrier substrate, wherein a side of the carrier substrate which faces the component and the opposite side of the component preferably form a fluid channel, wherein the at least one continuous opening of the carrier preferably communicates on its open side with the fluid channel. The component according to the invention is suitable for the installation and electrochemical measuring of transmembrane proteins, preferably in lipid bilayers. The invention also proposes different methods for producing the component.

The present invention relates to a component that is suitable for the examination of biological species, particularly for the electrochemical measuring and characterization of transmembrane proteins, having for that purpose a freestanding three-dimensionally formed, porous membrane structure with pores in the nanometer range, and a method for its production.

Generally, fine-pored structures made of glass or Teflon are used for measuring transmembrane proteins. For that purpose, a lipid bilayer is stretched over the frequently singular pore with diameters of a few μm to 150 μm, and the transmembrane proteins are subsequently introduced in the lipid bilayer. These pore-bearing structures are difficult to produce and thus expensive, and they can be used several times, and it must thus be possible to clean them. Therefore, such pore structures have for some time been made increasingly by means of semiconductor processes in silicon or other structurable materials, wherein the required electrodes can be integrated as microelectrode structures. This is advantageous because these “pore chips” can be designed as a relatively cost-effective disposable. One disadvantage is that the smaller the pore, the thinner the membrane that carries them must be in order to maintain the pore character and to not produce a “thin tunnel.” The pore-bearing structures thus become unstable because small pore geometries are required.

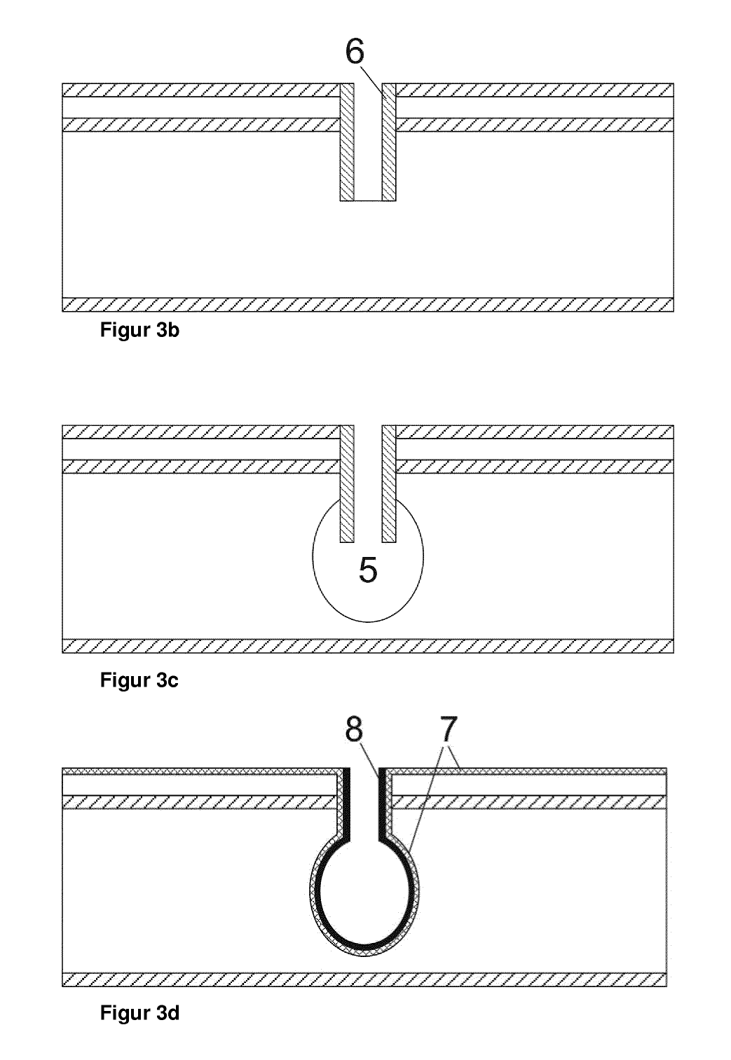

In the article “A chip-based biosensor for the functional analysis of single ion channels” by C. Schmidt, M. Mayer, H. Vogel, Angew. Chem. Int. Ed., 2000, 39, no. 17, 3137-3140, the production of a planar membrane with pores in the μm range is described. It is produced by creating an opening down to the Si₃N₄ layer in an Si-chip coated with Si₃N₄, through a combination of anisotropic KOH silicon etching and reactive ion etching, said opening being exposed and subsequently provided with one or a few pores by means of an etching process. Then, SiO₂ is applied from the gas phase onto the remaining surfaces. The chip is finally embedded in PDMS and the pores are covered with a lipid membrane. However, the number of pores remains small. FIG. 2 shows schematically the structure of such an arrangement.

C. Striemer et al., reports in “Charge- and size-based separation of macromolecules using ultrathin silicon membranes,” Nature Letters, vol. 445, 15 Feb. 2007, 749-753, on the production of a porous silicon membrane, having a multiplicity of pores with diameters in the range of 9 to 35 nm, and which is suitable for the charge- and size-based separation of biological or organic macromolecules. The method comprises the thermal deposition on both sides of an SiO₂ layer on a silicon substrate; the removal of a part on the rear side and the complete removal of the oxide on the front side; and the deposition of a triple layer of oxide/a-silicon/oxide with an a-silicon layer with 15 nm thickness, wherein a-silicon stands for amorphous silicon; a quick tempering step, during which the a-silicon is converted into nanocrystalline silicon under spontaneous formation of the nanometer pores, and the exposure of this layer through etching away of the oxide layers. The resulting silicon membrane has a multiplicity of irregularly distributed micropores. However, the disadvantage is the unfavorable volume-surface ratio of this chip. If the membrane is enlarged, its stability decreases. In addition, the fluidic coupling of volume-micromechanically produced components and their integration in a system is complicated.

Thin polymer membranes with periodically arranged pores can be produced by means of self-assembling structures using the so-called breath figures method, as described for example in “The influencing factors on the macroporous formation in polymer films by water droplet templating” by J. Peng, Y. Han, Y. Yang, B. Li, Polymer, 45 (2004) 447-452. The method is based on the ordered condensation of monodisperse water droplets on a thin polymer solvent layer in humid atmosphere. After the evaporation of water and solvent, a polymer film remains with a pore pattern which corresponds to the “imprints” of the droplets. An overview of the different variations can be found in “Advances in fabrication materials of honeycomb structure films by the breath-figure method” by L. Heng, B. Wang, M. Li, Y. Zhang, and L. Jiang, Materials, 6 (2013), 460-482. Alternatively, the self-organization of colloidal monodisperse particles into two-dimensional arrays can be used for producing a porous membrane during their sedimentation from a dispersion or under the influence of capillary forces, as described, for example, by K. Nagayama in “Two-dimensional self-assembly of colloids in thin liquid films,” Colloids and Surfaces A, 109 (1996) 363-374. Initially developed for photonic crystals, the method was used in “Electrochemical deposition of macroporous platinum, palladium and cobalt films using polystyrene latex sphere templates” by P. N. Bartlett, P. R. Birkin, and M. A. Ghanem, Chem. Commun., 2000, 1671-1672, for producing a metal layer with periodically arranged pores.

In “Rapid fabrication of nanoporous membrane arrays and single-pore membranes from parylene C†” by R. Thakar, R. Zakari, C. A. Morris, and L. A. Baker, Anal. Methods, 4 (2012), 4353-4359, a membrane made of parylene C with periodically arranged sub-μm pores is produced by coating a copper grid with much bigger openings (diameters of 10 μm and more). Owing to the high conformity of the parylene deposition, size and form of the resulting pores are very well defined.

In the article “Fully integrated micro Coriolis mass flow sensor operating at atmospheric pressure” by R. J. Wiegerink et al., Proc. MEMS Conf., Cancun, Mexico, 2011 Jan. 23-27, 1135-1138, a mass flow sensor is introduced, the fluidic channel of which is produced in the substrate and subsequently exposed through partial etching away of the substrate.

With similar techniques, hollow, almost spherical particles made of parylene were produced by Y. Xie, N. Banerjee, C. H. Mastrangelo as is described by these authors in “Microfabricated spherical pressure sensing particles for pressure and flow mapping,” Proc. Transducers Conf., Barcelona, Spain, 2013 Jun. 16-20, 1771-1774. They were initially formed within a recess in a silicon substrate and subsequently completely detached therefrom.

The problem addressed by the present invention is that of providing a membrane, having pores with diameters in the nanometer range, and despite the required extremely small thickness (as a rule, approximately 100 nm to 2 μm), having a high stability, which is integral part of a component, wherein the production of the component is supposed to be designed such that microelectrodes can be produced in a simple manner in close proximity of the membranes and thus the pores present therein in order to allow for very sensitive, spatially resolved electrochemical measurements, and wherein the component can be integrated without problems in larger components or other larger structures or assemblies with further, possibly complex structures, and so a coupling to fluidic systems is readily possible.

For solving this problem, the present invention provides a freestanding three-dimensionally formed, porous membrane that can be fabricated by means of micromechanical techniques as integral part of a component. When compared to a planar membrane, it is advantageous due to the enlarged surface and increased stiffness because the individual 3D structure can be selected to be comparatively small. Furthermore, due to its three-dimensionality, the membrane structure protrudes from the surface of the component, and so can be easier reached by a liquid medium, thus facilitating interaction. The porous membrane is preferably formed from an inorganic material, alternatively in some embodiments from a synthetic organic polymer (a plastic) or latex. The pores themselves are preferably produced without lithographic processes, i.e. either immediately during the membrane deposition or by means of a suitable post-treatment. A plurality of membranes/membrane structures can be combined or arranged in one array.

The membrane is part of a component as defined in claim 1. The component comprises a carrier made of a suitable structurable material with at least one continuous opening, which is closed by the porous membrane, characterized in that the porous membrane protrudes from the surface surrounding the continuous opening, preferably by approximately 5 to 300 μm. It is advantageous if the component has at least one electrode or an electrode pair in the vicinity of the opening on the side facing away from the membrane. Said electrode can be arranged directly on the surface of the component surrounding the opening or on an intermediate layer positioned on said surface.

The component can comprise an array of a plurality of randomly arranged openings, each one of which, as described above, is closed by a porous membrane and possibly provided with electrodes (electrode pairs).

As a rule, the individual openings have a diameter of only a few micrometers, preferably in the range from 5 to 100 μm. The porous membrane can have a structure which protrudes from the surrounding surface of the component in the manner of a bubble (approximately egg-shaped or spherical). As a rule, the diameter of the porous membrane is in this case larger than that of the opening. Advantageous diameters for such membrane structures are in the range from approximately 5 μm to approximately 200 μm. The porous membrane can also be formed in the shape of a cylinder, cone, or pyramid and have rounded corners and/or edges. In such case, the form of the opening corresponds to the layout of the membrane. The extension of the porous membrane parallel to the surrounding surface can also be greater than the opening.

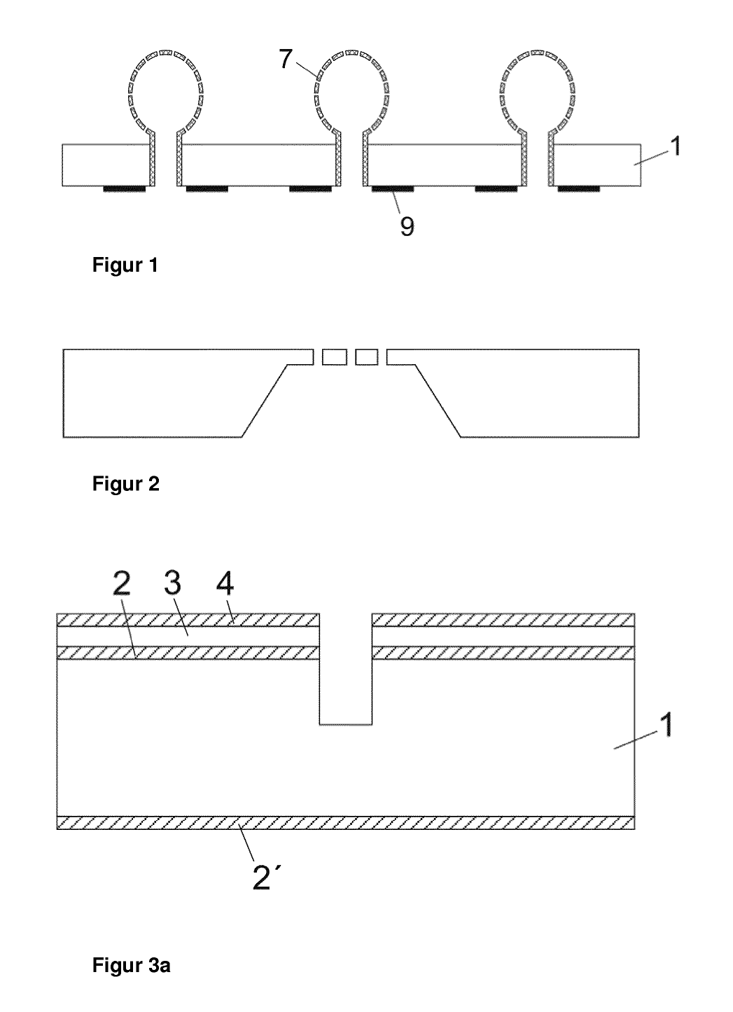

A schematic depiction of this component (here with an oval shape of the membrane) is shown in FIG. 1. The pores 7 of the membrane itself are in the nanometer range, i.e. they have an average diameter between 1 and 1000 nm, preferably between 50 and 1000 and more preferably between 50 and 500 nm at a membrane thickness of generally approximately 0.1 to 2 μm. The substrate is denoted with 1, reference sign 9 denotes the electrodes arranged on both sides of the openings.

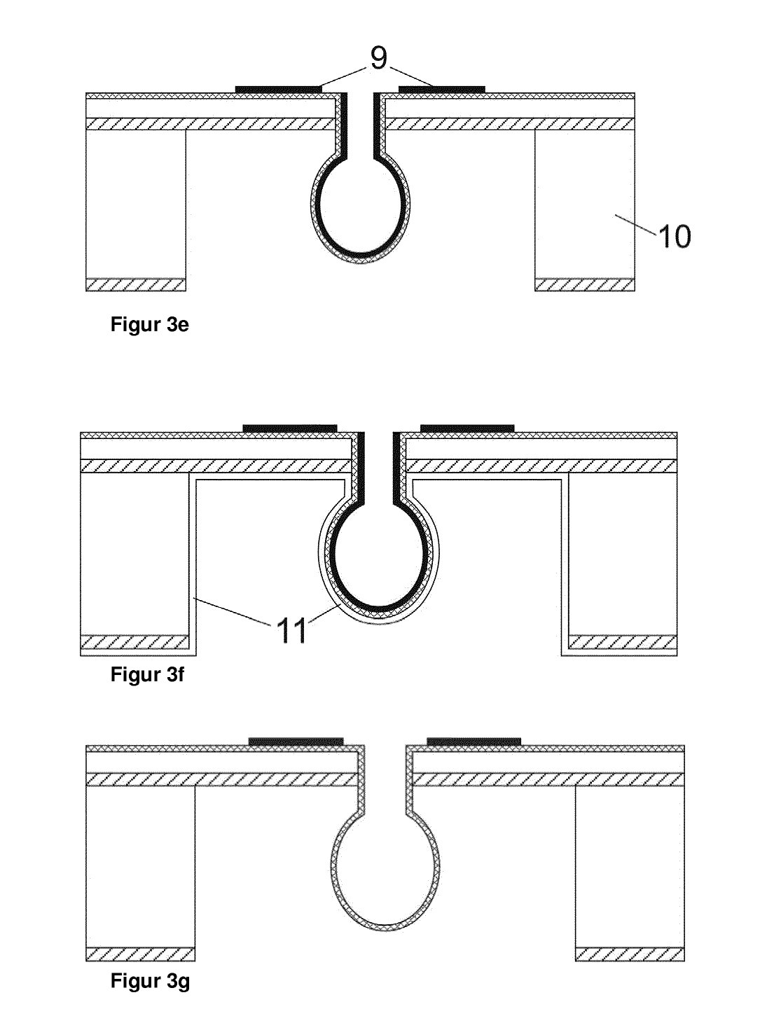

The membrane structures or the components provided with them can be obtained by proceeding from a structurable planar substrate, such as a silicon wafer or a silicon chip. With reference to FIGS. 3a to g, 4, and 5a to c, which are vertical sectional drawings through the respective constructs, this shall be explained in the following in more detail. It must be noted that, contrary to FIG. 1, the pores in the membrane in FIGS. 3, 5, and 6 are not depicted for simplification.

At first, the material that subsequently is supposed to serve as carrier for the 3D membrane structure is deposited with standard techniques on the substrate. This, for example, can be an oxide/poly-Si/oxide stack. It should be possible to etch the substrate material with high selectivity to the carrier material. In case of an oxide/poly-Si/oxide stack, this is ensured with the oxide layers, while the poly-Si provides the mechanical strength. The thickness of the poly-Si should thus be generally between approximately 5 and 100 μm, preferably between 10 and 50 min. Poly-Si layers with such a thickness can be produced by means of special CVD processes, for example, in the epitaxial reactor at temperatures of 900-1000° C. The thickness of the oxide layers, produced by means of conventional CVD processes, for example LPCVD (low pressure chemical vapor deposition), lies preferably in the range between 0.2 and 1 μm. Instead of oxide, it is also possible to use nitride or oxynitride, for example in the form of a nitride/poly-Si/nitride stack. The oxides, nitrides, and oxynitrides can be those from silicon, or they can be metal oxides, metal nitrides, or metal oxynitrides. Through RIE (reactive ion etching) by means of a conventional resist mask (first lithography step), a blind hole is subsequently etched through this stack (the carrier) and a part of the substrate below into the substrate. FIG. 3a shows the product of those two steps; the silicon substrate used therein is provided on its upper side with a triple-layer sequence (oxide/nitride 2, poly-Si 3, oxide/nitride 4, wherein the oxide and/or the nitride preferably is a compound of silicon), and on its rear side, it is also provided with a (silicon) oxide or (silicon) nitride layer 2′ which, however, at this point in time does not necessarily have to present. It must be noted that identical reference signs shall be used in all the following drawings.

After the removal of the resist (completion of the lithography step for forming the blind hole) and one or more optional cleaning processes for cleaning the substrate primarily from organic contaminants, for example, by means of an RCA cleaning, a protective layer, for example, an oxide, is deposited in the blind hole by means of a suitable method, preferably a CVD and particularly an LPCVD method, which completely lines the blind hole. For example, this is possible with a CVD method using TEOS (tetraethoxysilane), wherein silicon oxide is deposited. Subsequently, the protective layer is removed at least from the floor of the blind hole. If the protective layer is an oxide, this is possible, for example by means of anisotropic etching using RIE, and so the protective layer remains intact only on the (mostly vertical) walls of the blind hole. The product of these further method steps is shown in FIG. 3b, wherein the silicon oxide protective layer is denoted with reference sign 6.

At this point, the shape of the future membrane is determined or prepared. It can correspond to the walls of the blind hole or further etching steps can be executed, with which further material is removed from the substrate in the surroundings of the blind hole. As a rule, this is an isotropic etching process, with which, for example, an undercutting of the blind hole as well as rounded forms are achievable which, with regard to the form of the future membrane, is advantageous. Conventional etch gases for silicon in the IC technology, for example, one of the gases SF₆, CF₄, and CHF₃ as well as a mixture of two or more of these gases are suitable as etch gas hereto. The product of this step, i.e. the forming of a form deviating from the blind hole for the future membrane, is schematically shown in FIG. 3c. The blind hole (which is rounded in this embodiment) is denoted with reference sign 5.

After determining the form of the blind hole, it is lined isotropically with a layer which plays a key role for the further course of action in a first significant embodiment of the invention. Either this layer is subsequently transferred to the porous membrane, or it serves as an auxiliary layer for producing the porous membrane. In a number of important embodiments, which will be described in the following in more detail, this layer is a silicon oxide layer. Provided that the blind hole was not widened through etching and the silicon oxide protective layer 6 is still present in its entirety, the layer 6 can be used for this purpose. As a rule and particularly if the blind hole has been given its final shape only with the aforementioned etching step (wherein the oxide, due to its use as etch masking, naturally remains only in the neck regions of the blind hole which have not yet been etched), this oxide is removed and a new oxide layer is conformably deposited in the possibly widened blind hole and completely lines the etched cavity, preferably with a thickness of 0.1 to 2 μm. For this purpose, for example, the above-mentioned LPCVD method can be used. However, if the porous membrane is supposed to be formed from a different material than silicon oxide, the blind hole, after the removal of the oxide layer 6, can in this step be lined isotropically with such a material which is selected as required and in due consideration of the different applicable methods for producing the pores.

A person skilled in the art knows a multiplicity of methods for producing pores in thin layers, and these can be used on their merits without reservations, wherein, of course, the respective conditions (e.g. the tolerance of the other components for the temperature, with which the selected method has to be at least executed) must be taken into account. Examples for materials that can be transferred to a porous membrane or can serve as auxiliary layer thereto are, in addition to silicon oxide, CVD-deposited poly-Si or silicon nitride, metals such as aluminum or gold, which are applied through sputtering or galvanically from a liquid phase or through ALD (atomic layer deposition), as well as organic polymers, such as polystyrene or parylenes.

As required, the deposition of further layers, for example, a temporary support layer for stiffening the 3D structure prior to and/or during the forming of the porous membrane, or a layer which is selected as auxiliary layer for the subsequent production of the pores in the membrane material, and/or an etch protective layer during the exposure of the 3D structure, can be necessary or advantageous and follow the deposition of the aforementioned layer. For example, the temporary support layer can be poly-Si, deposited by means of LPCVD, or a metal, deposited through sputtering, CVD, or from the liquid phase, for example, through galvanic deposition. In addition to poly-Si or silicon oxide, different metal oxides, deposited by means of ALD, such as Al₂O₃, TiO₂, ZrO₂, can be used as etch protective layer. For the forming of pores in the membrane, poly-Si, deposited by means of LPCVD, or a metal layer, deposited by means of sputtering, can also be required. In all cases, it is required that the deposition process ensures a complete and preferably conform lining of the 3D cavity produced in the substrate. If applicable, the temporary protective layer and/or the other auxiliary layers are subsequently removed from the substrate surface using a conventional resist mask in a second lithography step by means of appropriate etching processes. For reasons of simplification, FIG. 3d, in addition to the material 7 which lines the 3D structure (the layer that subsequently is transferred in the porous membrane and serves as auxiliary layer for that purpose and which in many embodiments consists of silicon oxide) shows only one temporary support layer 8.

In a preferred embodiment of the invention, which can be combined with all other embodiments (and also those that will be explained further down), suitable metal electrodes are deposited and structured (for example, from a metal such as Pt, Au, Ir) on the carrier surface, preferably in the vicinity of the blind hole opening, which is shown in FIG. 3e (where the electrodes are denoted with reference sign 9). The metal electrode(s) can, for example, be produced by means of a liftoff process. For that purpose, a resist mask is applied to the substrate (or the upper SiO₂ layer of the carrier material stack). Then, the metal is deposited, for example by means of evaporation. The substrate is subsequently exposed to a solvent. The resist mask is dissolved and the metal located thereon is removed from the substrate. In the places where there was no resist, the metal remains on the surface. This step is called “third lithography step.” The deposition of the metal electrodes preferably takes place after the forming of the previously described layers and prior to the exposure of the 3D structure as will be explained in the following; however, this step can also take place prior to or even after the exposure of the 3D structure.

In the next method step, the 3D structure is exposed. For that purpose, the material of the substrate which surrounds the 3D structure, i.e. silicon in case of a silicon chip or wafer, must be removed. For that purpose, on the rear side of the substrate, which as a rule is already covered with an oxide or nitride layer, an etch opening is defined (this step is called the fourth lithography step), or the entire substrate surface is exposed. The front side is passivated by a suitable protective layer, for example, a photoresist. The material of the substrate is subsequently etched away in the desired places by means of known methods. In case of Si as a substrate, this can be achieved by means of DRIE (deep reactive ion etching) and/or in a XeF₂ gas phase. The exposed 3D structures remain anchored in the thick poly-Si layer (the carrier material) which was produced at the beginning of the process; see FIG. 3e. The initially present substrate can be completely etched away, or parts required for specific purposes can remain and can, for example, subsequently serve as carrier columns 10 or the like, as shown in FIG. 3e. The protective resist from the front side is preferably removed in the O₂ plasma. However, the use of solvents is also possible.

For producing pores in the now free-standing 3D structure, a polymer film 11 with an array of preferably monodisperse pores, which covers the entire rear side, can be produced by means of the breath-figure method; see FIG. 3f. The polymer can be applied, for example, from a solution, e.g. from a polystyrene solution in an organic solvent. Subsequently, the pores in the polymer film can be transferred through dry etching (reactive ion etching, RIE), as is standard in semiconductor technology, in the material of the 3D structure, which can be transferred into a porous membrane, wherein in this embodiment, it preferably consists of silicon oxide, but instead can also consist of a poly-Si or silicon nitride, deposited by means of CVD, or a metal such as aluminum or gold, deposited by means of sputtering, or through galvanic deposition from a liquid phase, or by means of ALD, or a stack of a plurality of layers of such or comparable materials one above the other. Depending on the process for transferring the material of the 3D structure into a porous membrane, the front side of the substrate will possible have to be passivated by a suitable protective layer, for example, a photoresist, in order to prevent damaging the structures and layers positioned thereon.

Instead of the porous polymer film in FIG. 3f, it is also possible to apply an array of monodisperse particles on the rear side. The particles, for example, can be made of an organic material, such as polystyrene (PS), polymethyl methacylate (PMMA), or latex, or an inorganic material, such as silicon oxide. The size of the particles should be selected such that the distance between adjacent particles in the array somewhat corresponds to the desired pore size. By means of dry etching (reactive ion etching, RIE), as is standard in semiconductor technology, the pore geometry of the particle array can be transferred into the material of the 3D structure. The material is once again preferably silicon oxide, but the materials mentioned in the previous paragraph can also be used.

In the last method step, the further present layer(s), i.e. the temporary support layer 8 and/or the auxiliary and/or the etch protective layer(s) are removed selectively to the porous membrane from the inner side of the 3D structure, and so only the porous membrane remains, as can be seen in FIG. 3g. The required etching processes must have a high selectivity relative to the membrane material. If the support layer is made of poly-Si, the membrane material can be removed by means of time-controlled etching in a XeF₂ gas phase. In this case, already exposed silicon surfaces will be slightly affected. In addition to silicon, XeF₂ etches only a few metals, such as Mo and W, and slightly also silicon nitride. However, silicon oxide, all other metals or metal oxides as well as organic materials are not etched. However, it is possible for the etch gas, particularly in case of longer processes, to penetrate organic materials. The porous polymer film is thus preferably also removed from the outer side of the 3D structure. This can be achieved by means of O₂ plasma. The transfer of the pore structure of the polymer film in FIG. 3f should preferably be executed from the gas phase or by means of a plasma.

The porous 3D structure can also be formed by an organic polymer film as such. In this case, for example, a polymer film 11 with an array of preferably monodisperse pores can be produced using the breath-figure method. Subsequently, both the temporary support layer 8 and the (in this case preferably used) silicon oxide 7 are removed by etching in the gas phase, and so only the porous polymer film 11 remains. In this design, the silicon oxide 7 serves as auxiliary layer for the forming of the future membrane.

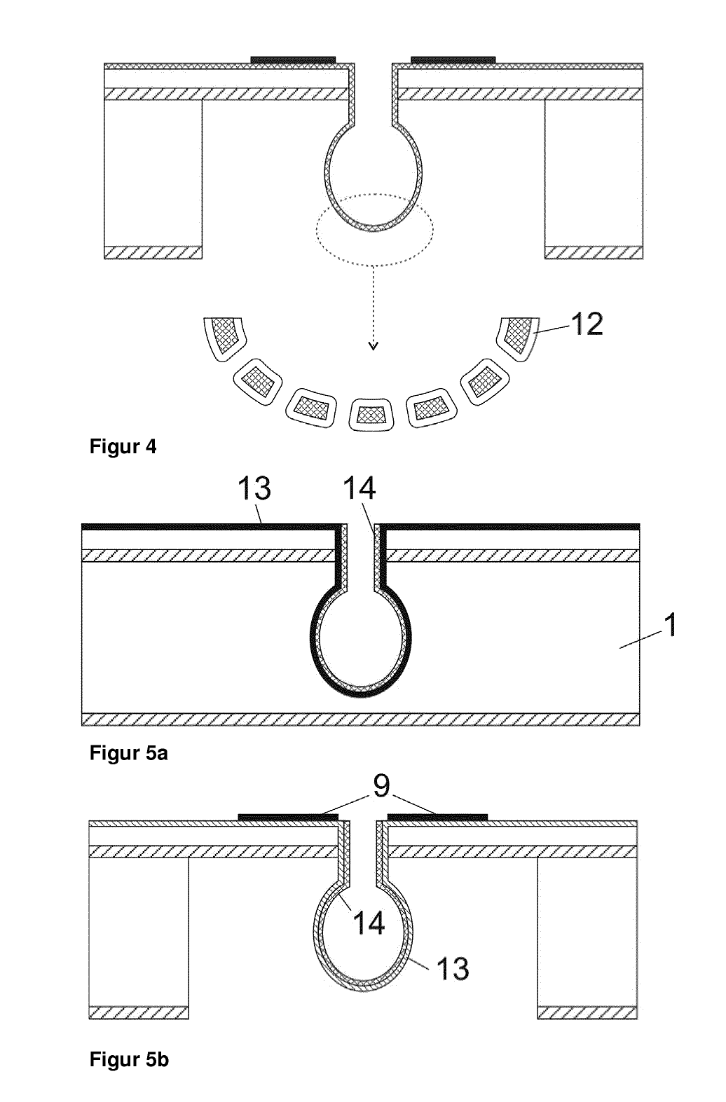

The size of the pores in the free-standing 3D structure, their mechanical stability and their physicochemical properties can be purposefully optimized by depositing further layers. The layers are deposited on the rear side of the substrate, from which the porous 3D structures protrude. Since the free-standing 3D structures are sensitive, processes are preferred which are characterized by a conformal coating from the gas phase at preferably low process temperatures. Examples are the deposition of parylene by means of CVD, or the deposition of metal oxides or nitrides by means of atomic layer deposition. The free-standing 3D structure is coated at least on the outer surface and in the pore openings, and so the diameter of the pores is evenly decreased. However, the 3D structure is preferably coated on all sides with a layer 12; see FIG. 4.

In an alternative embodiment, the 3D structure is made of metal, which is produced only after the exposure of the 3D structure on the silicon oxide layer, or such a metal is used for the forming of the pores. For such purpose, for example, an array of monodisperse particles, as already described, can be used as form for a galvanic deposition. A galvanic seed layer, e.g., made of gold, is applied, e.g. by means of sputtering, to the exposed 3D structure according to FIG. 3e. Subsequently, the particle array is produced on the galvanic seed layer, analogous to the polymer film as described for FIG. 3f. After the galvanic deposition of a suitable metal, such as gold, nickel, or copper, in the gaps of the particle array, the particles are removed in a suitable solvent. The galvanic seed layer must subsequently be etched out of the pores. Then, the pore structure can be transferred into the silicon oxide of the 3D structure by means of dry etching (reactive ion etching, RIE), as is standard in semiconductor technology. Alternatively, the dry etching can be foregone and after the removal of the temporary support layer 8, the silicon oxide, which in this case once again served as auxiliary layer for the forming of the future membrane, can also be etched away. For example, the oxide can be removed in the HF gas phase with high selectivity to many other materials. In addition to silicon oxide, only silicon nitride is affected in this gas phase; however, poly-Si, metals, or metal oxides are not affected. Organic materials are also not etched. However, in case of longer etching processes, it is possible for the HF to penetrate some organic materials which can result in defects (cracks, delamination). For that reason, such a combination is less advantageous.

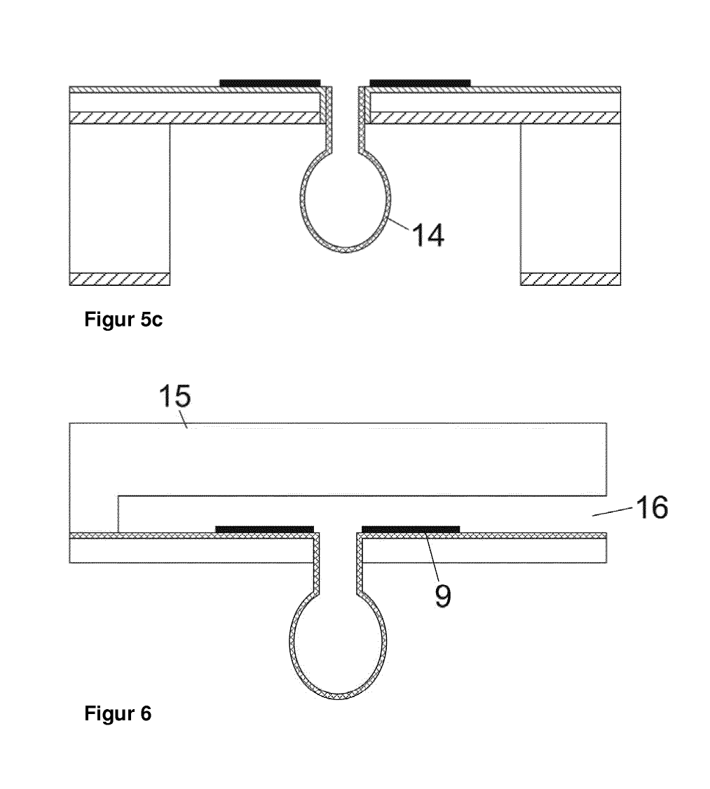

In a second, fundamentally different embodiment of the method, the porous membrane is not produced only after the exposure of the 3D structure from the rear side but as part of the layer sequence which lines the recess in the substrate according to FIG. 3d. This is advantageous because the production conditions of the membrane material are less limited, since no sensitive free-standing 3D structure is present. For example, much higher temperatures are possible. In this case, in the process sequence according to FIG. 3d, silicon oxide as temporary support layer 13 is preferably deposited first, followed by the actual membrane material 14 with a suitable thickness, preferably with a thickness of 0.1 to 2 μm, as is shown in FIG. 5a. In a preferred embodiment, it is already intrinsically porous. Intrinsically porous are, for example, thin poly-Si layers which were deposited in the epitaxial reactor at temperatures of 900-1000° C. Many dielectrics as well as metal layers are nanoporous when deposited at low temperatures (at up to 250° C.). The nanopores can be widened by means of etching. In aluminum, pores can be produced through anodic oxidation. For anodization, a conducting auxiliary layer, e.g. made of gold, is required below the aluminum.

After the forming of the porous membrane as inner layer, the 3D structure is exposed in an analogous manner, similar to the previously described embodiments. FIG. 5b shows the component after completion of the 3D structure, analogously to FIG. 3e. On the outer side, the porous membrane 14 is still covered by the silicon oxide layer 13, the first material, with which the blind hole, which determines the form of the 3D structure, was lined.

If the pores were produced through anodic oxidation in aluminum, the thereto required auxiliary layer can subsequently be removed through wet chemical etching in a suitable solvent. Alternatively, the pores can also be transferred to the auxiliary layer, e.g., by means of a dry etching process. The pores already present in the anodized aluminum layer serve as masking.

The completed component after removal of the silicon oxide is shown in FIG. 5c. As already described, the oxide 13 can be removed, for example, in an HF gas phase.

If no carrier columns or the like are needed for the future completed component, it can also be entirely removed or thinned mechanically. In such case, the substrate is thinned on the rear side according to FIG. 3e and FIG. 5b by grinding and polishing to the required thickness prior to producing the masking (fourth lithography step). It can also be removed entirely through further grinding/polishing and final etching of the entire surface. In this case, the masking is omitted.

In some embodiment of the invention, it is desirable to provide or form fluid channels over the opening(s) which are closed by the membrane. FIG. 6 shows such an embodiment. In these cases, a carrier substrate 15 is advantageously applied to and mounted on the front side of the carrier, for example, through bonding by means of an adhesive layer, before the silicon substrate is partially or completely removed. In this carrier substrate, one or more fluidic channels can be integrated, or such channels 16 can be formed between the front side of the coated polysilicon stack and the carrier substrate. It should be understood that each membrane structure on the wafer or chip, as e.g. shown in FIG. 1, can be provided with such a carrier substrate. Furthermore, instead of on the front side of the carrier, the electrodes 9 can also be located in the channels 16 of the carrier substrate 15.

Depending on the intended use, structures that were produced on entire wafers or larger chips and carry a multiplicity or a great number of porous membrane structures, are, if necessary, isolated, for example, by cutting up the wafer. In the isolated or individual complete component, it is also possible for only but also for a plurality of porous membrane structures to be present. As a result, either individual measurements or simultaneous multiparameter measurements or simultaneous measurements of different materials can be executed.

The components provided with a porous membrane according to the invention are suitable for electrochemical measurements and characterizations of the transmembrane proteins which are installed in the nanoporous 3D structures. Such a pore chip can be installed directly in a reactor, e.g., for cell-free protein synthesis because it is small, can be produced cost-efficiently, and is designed as disposable. The synthesized proteins are installed and measured directly in the lipid-carrying nanopores. Since the microstructured electrodes are directly adjacent to the pores, highly sensitive measurements are possible with the component according to the invention; due to the low production costs and the simple design, each component can be discarded after use and replaced by a new

1. A component comprising a carrier made of a structurable material with at least one continuous opening with a diameter of 5 to 100 μm and which is closed by a porous membrane, characterized in that the porous membrane with a thickness of 0.1 to 2 μm protrudes from the surface of the component surrounding the continuous opening.

2. The component of claim 1, wherein the carrier consists of at least two layers, wherein a first layer is made of an oxide or nitride or oxynitride, and a second layer is made of a polysilicon, and the porous membrane protrudes from the side of the surface of the component that is formed by the polysilicon layer.

3. The component of claim 2, wherein the carrier has at least one third layer which is located on the side of the polysilicon that faces away from the first layer.

4. The component of claim 2, further comprising a substrate made of a structurable material, preferably a silicon substrate, as well as an oxide, nitride, or oxynitride layer on the upper side of the substrate, said layer being located at least in certain sections in the direct or indirect vicinity of the polysilicon of the carrier.

5. The component of claim 2, wherein the oxide, or nitride, or oxynitride of the layer or the layers is a silicon oxide, silicon nitride, or silicon oxynitride.

6. The component of claim 1, comprising a multiplicity of continuous openings, which are each closed by a porous membrane, wherein at least a part of or all porous membranes protrude from the surface of the component surrounding the continuous opening and preferably have the same form and the same measurements.

7. The component of claim 1, wherein at least one electrode is located on the side of the component that faces away from the membrane in the vicinity of the at least one continuous opening or pore.

8. The component of claim 1, wherein the porous membrane is made of silicon, silicon oxide, aluminum oxide, a metal coated with a metal oxide, a metal, particularly aluminum or gold, silicon nitride, polystyrene, polymethyl methacylate (PMMA), latex, or parylene.

9. The component of claim 1, wherein the porous membrane has pores, the diameter of which lies on average in the range between 50 and 1000 nm.

10. The component of claim 1, wherein the component further comprises a carrier substrate, wherein a side of the carrier substrate which faces the component and the opposite side of the component preferably form a fluid channel, wherein the at least one continuous opening of the carrier preferably communicates on its open side with the fluid channel.

11. (canceled)

12. A method for producing the component of claim 1, comprising the following steps:
(a) Provision of a substrate made of a structurable material, on the front side of which a carrier, which is also made of a structurable material, is located;
(b) Application of a mask on the front side of the carrier and etching of a blind hole through the carrier into the substrate at the place where the at least one opening is eventually supposed to be located;
(c) isotropic and conform precipitation of a layer made of a material in the blind hole which is provided for the transfer to a porous membrane or as auxiliary layer for the production of said porous membrane;
(d) if so desired, isotropic and conform precipitation of at least one further layer on the layer of the material that is provided for the transfer in a porous membrane or as auxiliary layer for the production of said porous membrane, selected from support layers, etching layers, and auxiliary layers for producing pores in the layer applied according to step (c);
(e) Exposure of the material that is provided for the transfer to a porous membrane or as auxiliary layer for the production of said porous membrane, by etching away of rear-side substrate material, comprising the passivating of the front side of the carrier and those areas on the rear side of the substrate that are not supposed to be affected, and etching of the substrate material by means of DRIE or XeF₂ gas;
(f) Production of pores in the layer of the material that is provided for the transfer to a porous membrane, or production of a porous layer on the layer that is provided as auxiliary layer;
(g) if necessary, removal of the layer or layers that were precipitated according to step (d), and/or the auxiliary layer.

13. The method of claim 12, wherein the material of the layer, which is precipitated according to step (c) as well as step (f), is selected from one of the following combinations (i) to (iv):
(i) the material of the layer which is precipitated according to step (c) is selected from silicon oxide, polysilicon, silicon nitride, or a metal, particularly aluminum or gold; step (f) comprises: the outside application of a layer made of an organic polymer, the production of pores in the organic polymer by means of the breath-figure method, and transfer of the pores in the material of the layer that was precipitated according to step (c) through dry etching;
(ii) the material of the layer which is precipitated according to step (c) is selected from silicon oxide, polysilicon, silicon nitride, or a metal, particularly aluminum or gold; step (f) comprises: the outside application of an array of monodisperse particles made of an organic or inorganic material such that the distances between the particles correspond to the desired pore size, and transfer of the pore geometry of the particle arrays in the layer that was precipitated according to step (c) through dry etching;
(iii) the material of the layer which is precipitated according to step (c) consists of silicon oxide; step (f) comprises: the outside application of a layer made of an organic polymer, the production of pores in the organic polymer by means of the breath-figure method, and the etching away of the silicon layer through gas phase etching;
(iv) the material of the layer which is precipitated according to step (c) consists of silicon oxide; step (f) comprises in the specified sequence the outside application of a galvanic seed layer, the application of an array of monodisperse particles on the galvanic seed layer, the galvanic precipitation of a metal, the removal of the particles by means of a solvent, the etching of the galvanic seed layer from the resulting pores, and either transfer of the pore structure in the silicon oxide layer through dry etching, or the etching away of the silicon oxide layer.

14. A method for producing the component of claim 1, comprising:
(a) Providing a substrate made of a structurable material, on the front side of which a carrier, which is also made of a structurable material, is located;
(b) Application of a mask on the front side of the carrier and etching of a blind hole through the carrier into the substrate at the place where the at least one opening is eventually supposed to be located;
(c′) isotropic and conform precipitation of a layer made of silicon oxide in the blind hole;
(d′) isotropic and conform precipitation of a porous layer or a non-porous layer on the silicon oxide layer, wherein in case of the precipitation of a non-porous layer, such layer is subsequently provided with pores;
(e′) Exposure of the silicon oxide layer, precipitated according to step (c′), by etching away rear-side substrate material, comprising the passivating of the front side of the carrier and those areas on the rear side of the substrate that are not supposed to be affected, and etching of the substrate material by means of DRIE or XeF₂ gas;
(g′) Etching away of the silicon oxide layer precipitated according to step (c′).

15. The method of claim 14, wherein step (d′) is selected from:
(i) precipitation of an intrinsically porous polysilicon layer in the epitaxial reactor at temperatures from 900-1000° C.;
(ii) precipitation of a nanoporous dielectric or metal layer at a temperature of ≦250° C.;
(iii) precipitation of a conducting metallic auxiliary layer followed by an aluminum layer, and production of pores in the aluminum layer through anodic oxidation.

16. The method of claim 15, wherein the carrier is produced on the substrate by the successive precipitation of a triple layer on the substrate, wherein the first layer consists of an oxide, a nitride, or an oxynitride of silicon or a metal, the second layer consists of polysilicon, and the third layer consists of an oxide.

17. The method of claim 12, wherein the method comprises the precipitation of metal electrodes on the front side of the carrier in the vicinity of the at least one continuous opening, characterized in that the respective metal is precipitated after application of a mask, and that the mask is subsequently wet-chemically removed.

18. The method of claim 17, wherein metal electrodes are formed according to step (d).

19. The method of claim 12, wherein the method further comprises after completion of step (b) and prior to step (c), the application of a protective layer in the blind hole, and the removal of this protective layer from the floor surface of the blind hole and possibly from adjacent areas, and execution of an isotropic etch step by enlarging the blind hole,
and/or,
after completion of the last method step according to claim 12, the thinning of the (remaining) rear-side substrate material and/or
(k) application of a carrier substrate on the component, wherein a side of the carrier substrate which faces the component and the opposite side of the component form a fluid channel.

20. The method of claim 12, wherein the method further comprises the modifying of the size of the pores in the membrane and/or the modifying of the mechanical stability and/or the physicochemical properties of the membrane through coating of at least the outer surface of the membrane and preferably the pore openings.

21. A method for use of a component of claim 1, comprising measuring electrochemical behavior of transmembrane protein in a lipid bilayer.
